(19) United States
(12) Patent Application Publication
(10) Pub. No.: US 2015/0087989 A1
(43) Pub. Date: Mar. 26, 2015

(54) ULTRASOUND PROBE AND ULTRASOUND SCANNING SYSTEM

(71) Applicants: QISDA (SUZHOU) CO., Ltd., Suzhou (CN); QISDA CORPORATION, Taoyuan County (TW)

(72) Inventors: Yu-Hsiu Lee, Taipei City (TW); Chih-Hung Chen, Taipei City (TW); Chun-Chieh Wang, New Taipei City (TW)

(21) Appl. No.: 14/318,704

(22) Filed: Jun. 30, 2014

(30) Foreign Application Priority Data
Sep. 24, 2013 (TW) 102134333

(51) Int. Cl. A61B 8/00

(52) U.S. Cl. CPC A61B 8/445; A61B 8/4483
USPC 600/459

(57) ABSTRACT

An ultrasound probe includes a casing, an ultrasound scanner and a passage. The ultrasound scanner is disposed in the casing. The passage is disposed on the casing and used for transporting an ultrasound conductive medium.

### BACKGROUND OF THE INVENTION

1. Field of the Invention

The present invention relates to an ultrasound probe and an ultrasound scanning system, and more particularly, to an ultrasound probe with a passage for transporting the ultrasound conductive medium and an ultrasound scanning system in which aforementioned ultrasound probe is applied.

2. Description of the Prior Art

The ultrasound probe is generally applied in the fields of materials and clinic medical inspection due to the characteristics that the material structures and the human cells would not be ruined. Since the preciseness of the ultrasound is affected by the air, before the inspection is performed via the ultrasound probe, the gel is normally applied on the inspected portion so as to facilitate the ultrasound conduction. Generally speaking, the operator must hold the gel bag, squeeze the gel from the bag, apply the gel on the inspected portion, and then use the ultrasound probe over the inspected portion to perform the ultrasound inspection. If the gel applied on the inspected portion is not enough, or the operator needs to inspect other portion, the operator will have to hold the gel bag, squeeze the gel from the bag, and apply the gel on the inspected portion again. It not only causes trouble but also decreases the inspection efficiency in the aforementioned way.

### SUMMARY OF THE INVENTION

Due to the problems stated in the description of the prior art, one purpose of the present invention is to provide an ultrasound probe with a passage for transporting the ultrasound conductive medium and an ultrasound scanning system in which aforementioned ultrasound probe is applied

According to one embodiment, an ultrasound probe of this invention comprises a casing, an ultrasound scanner and a passage. The ultrasound scanner is disposed in the casing. The passage is disposed on the casing and used for transporting an ultrasound conductive medium.

According to another embodiment, an ultrasound scanning system of the invention comprises an ultrasound scanning device, an ultrasound probe, a container and a driving device. The ultrasound probe communicates with the ultrasound scanning device. The ultrasound probe comprises a casing, an ultrasound scanner and a passage. The ultrasound scanner is disposed in the casing. The passage is disposed on the casing. The container is used for containing an ultrasound conductive medium. The driving device is connected between the passage and the container. The driving device is used for transporting the ultrasound conductive medium from the container to the passage and squeezing the ultrasound conductive medium out of the passage.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is an illustrative diagram of an ultrasound probe according to an embodiment of the present invention.

FIG. 2 is an illustrative diagram of an ultrasound scanning system in which the ultrasound probe shown in FIG. 1 is applied.

FIG. 3 is an illustrative diagram of an ultrasound probe according to another embodiment of the present invention.

FIG. 4 is an explosion diagram of part of the components shown in FIG. 3.

FIG. 5 is an illustrative diagram of an ultrasound probe according to another embodiment of the present invention.

FIG. 6 is an illustrative diagram of an ultrasound scanning system in which the ultrasound probe shown in FIG. 5 is applied.

FIG. 7 is an illustrative diagram of an ultrasound scanning system according to another embodiment of the present invention.

FIG. 8 is an illustrative diagram of an ultrasound probe according to another embodiment of the present invention.

FIG. 9 is an illustrative diagram of an ultrasound probe according to another embodiment of the present invention.

### DETAILED DESCRIPTION

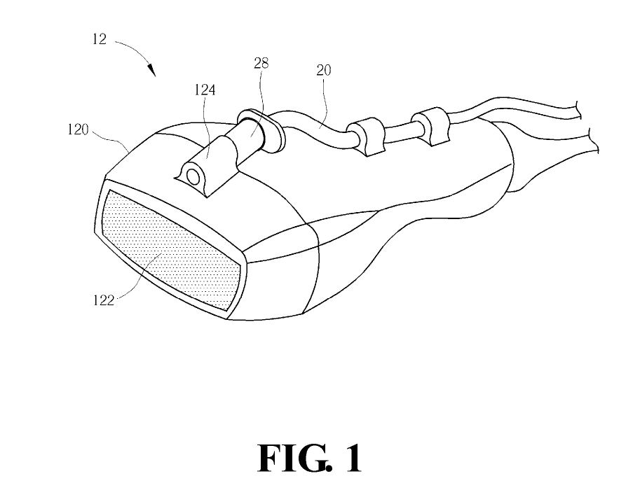

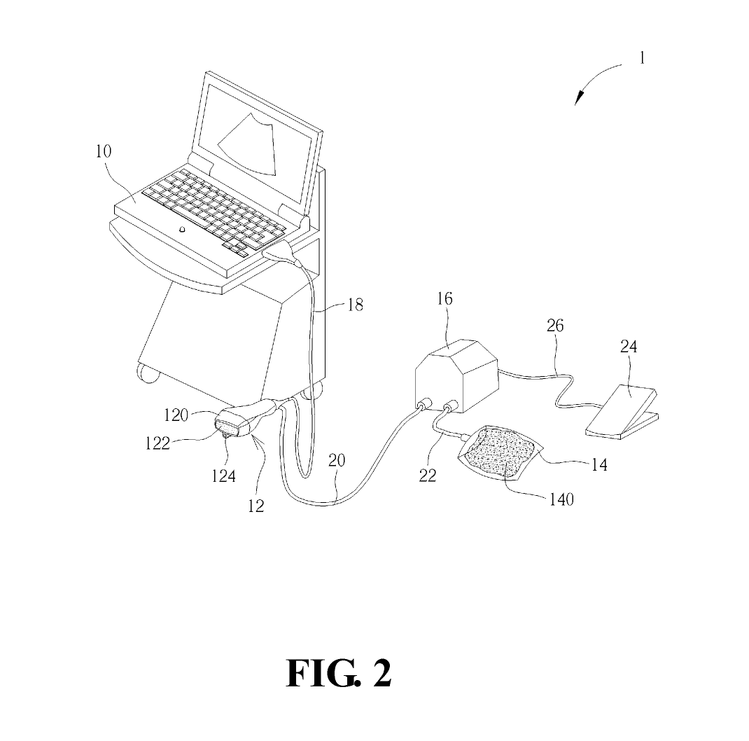

Please refer to FIG. 1 and FIG. 2. FIG. 1 is an illustrative diagram of an ultrasound probe according to an embodiment of the present invention. FIG. 2 is an illustrative diagram of an ultrasound scanning system in which the ultrasound probe shown in FIG. 1 is applied. As shown in FIG. 2, the ultrasound scanning system 1 comprises an ultrasound scanning device 10, an ultrasound probe 12, a container 14 and a driving device 16. The ultrasound scanning device 10 can be a computer or other electronic device with data calculation, processing and display functions. The ultrasound probe 12 and the ultrasound scanning device 10 communicate with each other via a cable 18 so that when the ultrasound probe 12 is applied on the inspected portion of a body, the display of the ultrasound scanning device 10 can display the corresponding image.

As shown in FIG. 1, the ultrasound probe 12 comprises a casing 120, an ultrasound scanner 122 and a passage 124. The ultrasound scanner 122 is disposed in the casing 120. The passage 124 is disposed on the casing 120. Please note that the operation and the structure of the ultrasound scanner 122 are well known by those of ordinary skill in the art, and therefore are not explained in details herein. In this embodiment, the passage 124 and the casing 120 are formed integrally, but are not so limited.

The container 14 is used to contain an ultrasound conductive medium 140, and the ultrasound conductive medium 140 can be water or soft medium, such as transparent gel. The ultrasound conductive medium 140 is to facilitate the ultrasound conduction. The driving device 16 is connected between the passage 124 of the ultrasound probe 12 and the container 14 by the pipes 10 and 22. In this embodiment, the driving device 16 is a pump used to transport the ultrasound conductive medium 140 from the container 14 to the passage 124 of the ultrasound probe 12 and then to squeeze the ultrasound conductive medium 140 out of the passage 124.

As shown in FIG. 2, the ultrasound scanning system further comprises a pedal 24, and the pedal 24 is connected to the driving device 16 by a signal line 26 so as to drive the driving device 16 to transport the ultrasound conductive medium 140 from the container 14 to the passage 124 of the ultrasound probe 12 and then to squeeze the ultrasound conductive medium 140 out of the passage 124.

Therefore, when the ultrasound inspection is performed by the ultrasound probe 12, the operator only needs to put the passage 124 of the ultrasound probe 12 closed to the inspected portion of the body and to step on the pedal 24, the driving device 16 is controlled to transport the ultrasound conductive medium 140 from the container 14 to the passage 124 of the ultrasound probe 12 via the pipes 22 and 20, and then to squeeze the ultrasound conductive medium 140 out of the passage 124 of the ultrasound probe 12. The ultrasound conductive medium 140 squeezed out of the passage 124 can then be applied on the inspected portion by the casing 120 of the ultrasound probe 12 directly for the ultrasound scanning over the inspected portion. When the ultrasound conductive medium 140 applied on the inspected portion is not enough or when the operator wants to check other inspected portion, the operator only needs to step on the pedal 24 again, and the driving device 16 is controlled to transport the ultrasound conductive medium 140 from the container 14 to the passage 124 of the ultrasound probe 12 via the pipes 22 and 20, and then to squeeze the ultrasound conductive medium 140 out of the passage 124 of the ultrasound probe 12 without putting down the ultrasound probe. Besides, when the operator steps on the pedal 24 to control the driving device 16, the operator's hand which does not hold the ultrasound probe 12 can operate or set up the ultrasound scanning device 10 upon the operator's need. In this way, it not only is more convenient and simple to perform the ultrasound inspection, but also increases the inspection efficiency.

In another embodiment of this invention, the pedal 24 is replaced by a control module (not shown). The control module communicates with the driving device 16 through wire or wireless communication. The operator operates the control module to drive the driving device 16 to transport the ultrasound conductive medium 140 from the container 14 to the passage 124 of the ultrasound probe 12 and then to squeeze the ultrasound conductive medium 140 out of the passage 124. In this embodiment, a rapid connector 28 is disposed in the front end of the pipe 20 as shown in FIG. 1. Through the rapid connector 28, the pipe 20 can be rapidly connected to and easily detached from the passage 124 of the ultrasound probe 12.

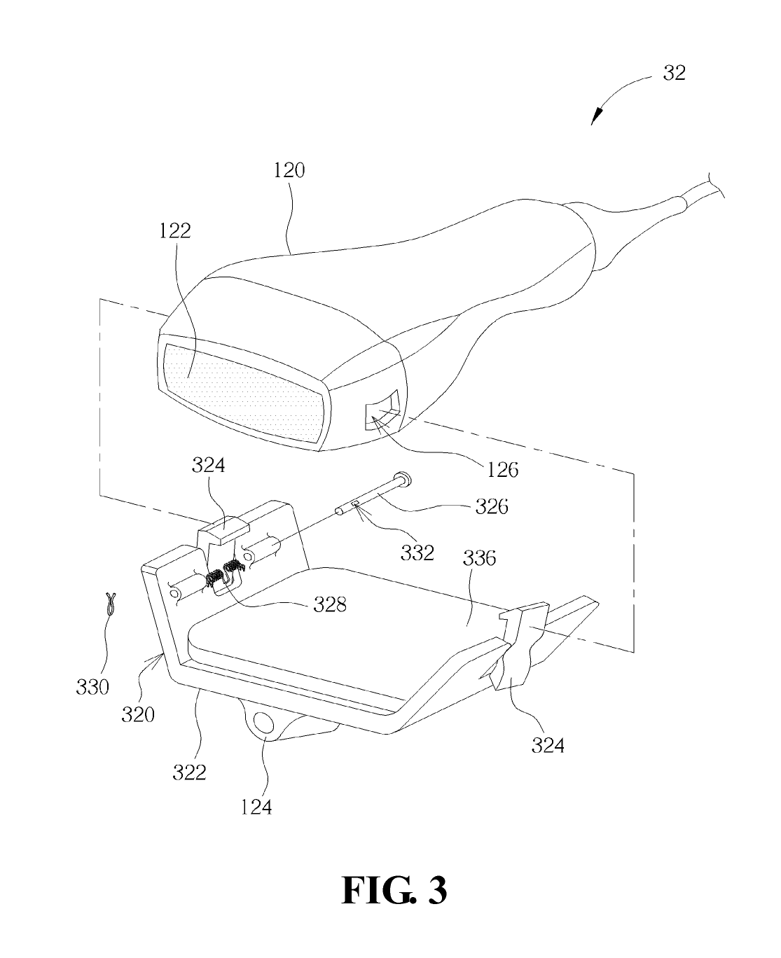

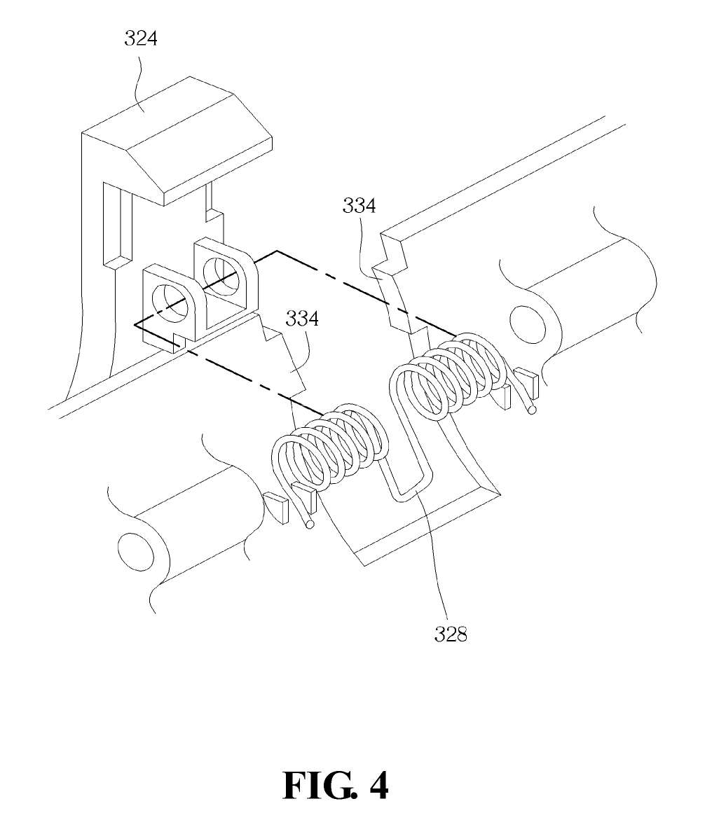

Please refer to FIG. 3 and FIG. 4. FIG. 3 is an illustrative diagram of an ultrasound probe according to another embodiment of the present invention. FIG. 4 is an explosion diagram of part of the components shown in FIG. 3. The differences between the ultrasound probe 32 and the aforementioned ultrasound probe 12 are that the ultrasound probe further comprises a connection mechanism 320, that the connection mechanism 320 comprises a base 322 detachedly connected to the casing 120, and that the passage 124 and the base 322 are formed integrally. As shown in FIG. 3 and FIG. 4, the casing 120 has two slots 126, and the connection mechanism 320 further comprises two stuck members 324, two hinges 326, two torsion springs 328 and two latches 330. Due to the viewing angle, FIG. 3 and FIG. 4 show only the slot 126 on one side of the casing 120 as well as the hinge 326, the torsion spring 328 and the lath 330 on one side of the base 322. The stuck members 324 are hinged with the base 322 by the hinges 326. The torsion springs 328 are disposed on the hinges 326, and contact the base 322 and the stuck members 324, respectively. After the stuck members 324, the hinges 326 and the torsion springs 328 are assembled with the base 322, the latches 330 can pass through the fixing holes 332 of the hinges 326 to fix the assembly.

In this embodiment, the operator can press the casing 120 of the ultrasound probe 120 toward the base 322,so that the two stuck members 324 rotate apart from each other. At this time, the torsion springs 328 are twisted. When the casing 120 is press downward to the end, the torsion springs 328 generate torsion force to have the stuck members 324 return to where the stuck members 324 are originally disposed, so that the stuck members 324 are stuck with the slots 126 of the casing. In this way, the base 322 are connected to the casing 120 to have the passage 124 be disposed on the casing 120 via the base 322. When the operator desires to detach the base 322 from the casing 120, the operator simply presses the end of the stuck members 324, and the stuck members 324 then rotate to be released from the slots 126 of the casing 120. When the stuck members 324 are pressed, the torsion springs 328 are twisted. When the stuck members 324 are released, the torsion spring 328 generate torsion force to have the stuck members 324 return to where the stuck members 324 are originally disposed. As shown in FIG. 4, the base 322 comprises two stoppers 224 to limit rotation of the stuck members 324 in a predetermined range.

In addition, as shown in FIG. 3, the connection mechanism 320 further comprises an elastic member 336 disposed on the base 322. When the base 322 is connected to the casing 120, the elastic member 336 is clamped between the casing 120 and the base 322. In this way, shake of the base 322 and the casing 120 due to the mechanical tolerance can be avoided. In this embodiment, the elastic member 336 may be a rubber pad or a sponge pad, but is not so limited.

Please note that the base 322 may be detachedly disposed on the casing 120 by magnetism or screwing instead of aforementioned stuck members 324 and slots 126. For example, two magnets may be disposed on the base 322 and the casing 120, respectively; or the base 322 and the casing 120 have corresponding screw holes, and one screw passes through the screw holes of the base 322 and the casing 120 for fixing. Please not that the components shown in FIG. 3 and FIG. 4 with the same labeling numbers as those shown in FIG. 1 and FIG. 2 operate in substantially the same way and are not repeatedly described in details herein.

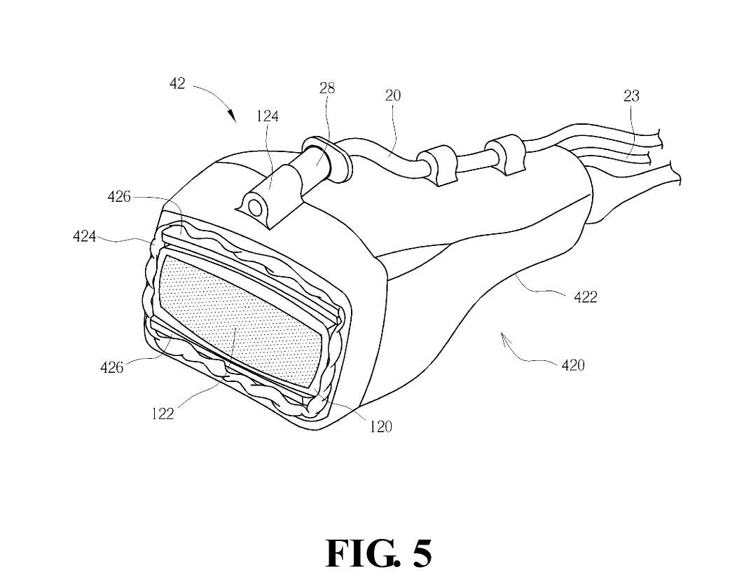

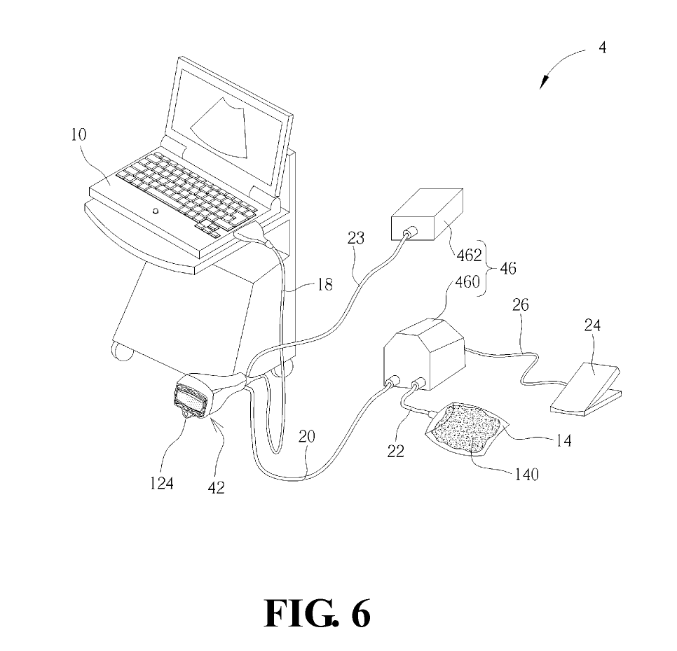

Please refer to FIG. 5 and FIG. 6. FIG. 5 is an illustrative diagram of an ultrasound probe according to another embodiment of the present invention. FIG. 6 is an illustrative diagram of an ultrasound scanning system in which the ultrasound probe shown in FIG. 5 is applied. The differences between the ultrasound probe 42 and the aforementioned ultrasound probe 12 are that the ultrasound probe 43 further comprises a connection mechanism 420, that the connection mechanism 420 comprises a base 422 detachedly connected to the casing 120 and that the passage 124 and the base 422 are formed integrally. As shown in FIG. 5, the base 433 is ring-shaped hollow. Besides, the connection mechanism 420 further comprises a ring-shaped hollow air bag 424 disposed in the base 422. As shown in FIG. 6, the driving device 46 of the ultrasound probe 42 comprises a first pump 460 and a second pump 462. The first pump 460 is connected to the passage 124 and the container 14 by the pipes 20 and 22. The second pump 462 is connected to the ring-shaped hollow air bag 424 by the pipe 23. In this embodiment, the first pump 460 is used to transport the ultrasound conductive medium 140 from the container 14 to the passage 124 and then to squeeze the ultrasound conductive medium 140 out of the passage 124. The second pump 462 is used to activate air inflation or leakage of the ring-shaped hollow air bag.

In this embodiment, the operator can have the casing 120 of the ultrasound probe 42 pass through and be surrounded by the ring-shaped hollow air bag 424, and then drives the second pump 462 to activate air inflation of the ring-shaped hollow air bag 424. After the casing 120 of the ultrasound probe 42 is surrounded by the ring-shaped hollow air bag 424 and the ring-shaped hollow air bag 424 is full of air, the casing 120 is clamped in the base 422 by the ring-shaped hollow air bag 424 so that the passage 124 is disposed on the casing 120 by the base 422. In this embodiment, the connection mechanism 420 further comprises two elastic members 426 disposed on the inner sides of the top and bottom surfaces of the ring-shaped hollow air bag 424. After the casing 120 passes through and is then surrounded by the ring-shaped hollow air bag 424 and the ring-shaped hollow air bag 424 is full of air, the elastic members 426 contact the casing 120 so as to have the casing 120 be tightly clamped by the ring-shaped hollow air bag 424. Please not that the components shown in FIG. 5 and FIG. 6 with the same labeling numbers as those shown in FIG. 1 and FIG. 2 operate in substantially the same way and are not repeatedly described in details herein.

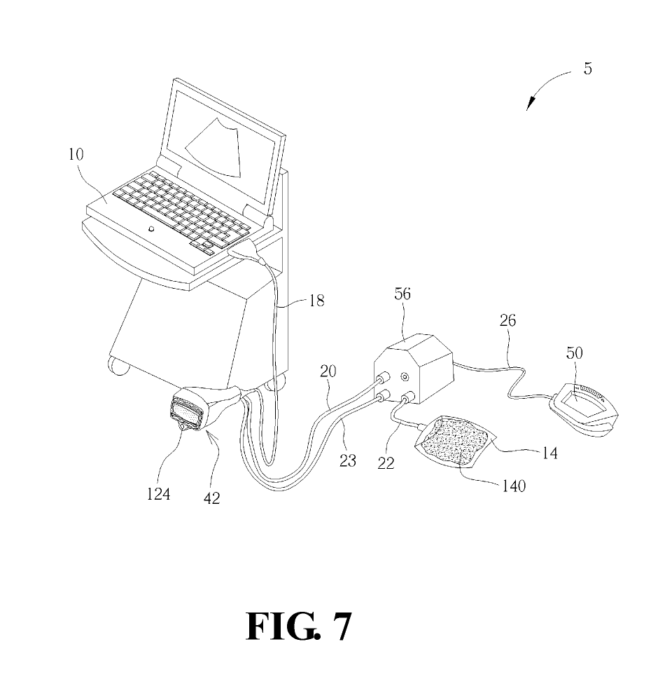

Please refer to FIG. 7. FIG. 7 is an illustrative diagram of an ultrasound scanning system 5 according to another embodiment of the present invention. The differences between the ultrasound scanning system 5 and aforementioned ultrasound scanning system 4 are that in the ultrasound scanning system 5 the passage 124, the container 14 and the ring-shaped hollow air bag 424 are connected to the single driving device 56 by the pipes 20, 22 and 23. In other words, in the invention it is able to use a single driving device 56 to transport the ultrasound conductive medium 140 from the container 14 to the passage 124 through the pipes 22and 20, and to activate air inflation or leakage of the ring-shaped hollow air bag through the pipe 23. In addition, in the ultrasound scanning system 5 the control module 50 is used to communicate with the driving device 56 via wire or wireless communication so that the operator operates the control module 50 to drive the driving device 56 to transport the ultrasound conductive medium 140 from the container 14 to the passage 124 through the pipes 22 and 20, and to activate air inflation or leakage of the ring-shaped hollow air bag 424 through the pipe 23. Please not that the components shown in FIG. 7 with the same labeling numbers as those shown in FIG. 6 operate in substantially the same way and are not repeatedly described in details herein.

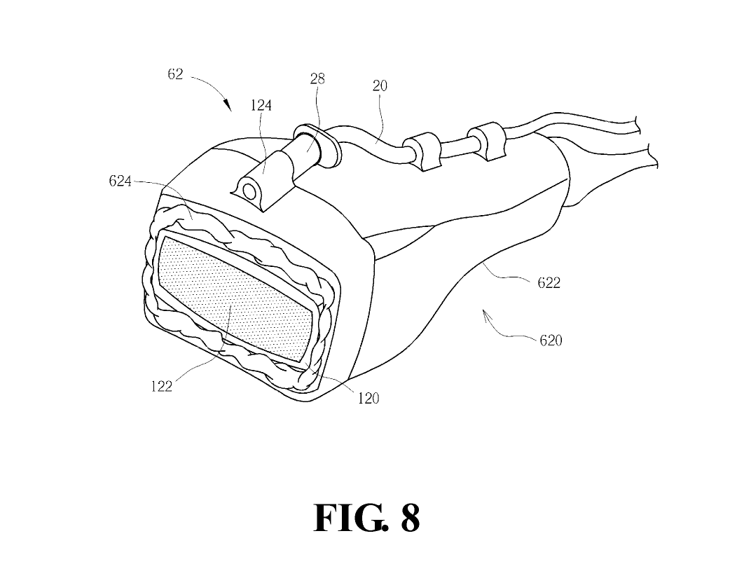

Please refer to FIG. 8. FIG. 8 is an illustrative diagram of an ultrasound probe according to another embodiment of the present invention. The differences between the ultrasound probe 62 and the ultrasound probe 12 are that the ultrasound probe 62 further comprises a connection mechanism 620, that the connection mechanism 620 comprises a base 622 detachedly connected to the casing 120, and that the passage 124 and the base 622 are formed integrally. As shown in FIG. 8, the base 622 is ring-shaped hollow. Besides, the connection mechanism 620 further comprises a ring-shaped hollow elastic member 624 disposed in the base 622. In this embodiment, the ring-shaped hollow elastic member 624 is made of rubber or sponge, but is not so limited. In this way, the casing 120 passes through and is then surrounded by the ring-shaped hollow elastic member 624 so as to have the casing 120 be clamped in the base 622 by the ring-shaped hollow elastic member 624. Please not that the components shown in FIG. 8 with the same labeling numbers as those shown in FIG. 1 operate in substantially the same way and are not repeatedly described in details herein.

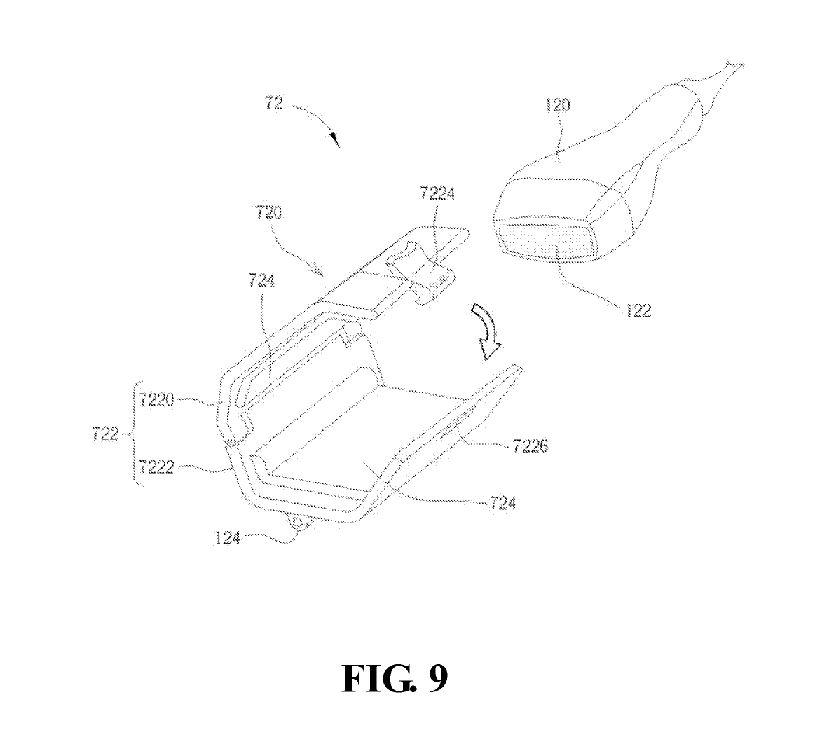

Please refer to FIG. 9. FIG. 9 is an illustrative diagram of an ultrasound probe according to another embodiment of the present invention. The differences between the ultrasound probe 72 and the ultrasound probe 12 are that the ultrasound probe 72 further comprises a connection mechanism 720, that the connection mechanism 720 comprises a base 722 detachedly connected to the casing 120, and that the passage 124 and the base 722 are formed integrally. As shown in FIG. 9, the base 722 comprises a top cover 7220 and a bottom cover 7222. One end of the top cover 7220 is hinged with the bottom cover 7222, and the other end of the top cover 7220 is selectively stuck with or released from the bottom cover 7222. In this embodiment, the passage 124 is formed on the bottom cover 7222, but is not so limited. A stuck member 7224 is disposed on the top cover 7220 and a slot 7226 is formed on the bottom 7226 so that the stuck member 7224 is selectively stuck with or released from the slot 7226. In addition, the connection mechanism 720 further comprises two elastic members 724 disposed on the inner side of the top cover 7220 and on the inner side of the bottom cover 7222, respectively. When the casing 120 is disposed between the top cover 7220 and the bottom cover 7222 and the stuck member 7224 of the top cover 7220 is stuck with the slot 7226 of the bottom cover 7222, the casing 120 is clamped between the top cover 7220 and the bottom cover 7222 by the two elastic members 724. In this way, the casing 120 is tightly held in the base 722 and shake of the base 722 and the casing 120 due to the mechanical tolerance can be avoided. In this embodiment, the elastic members 724 may be rubber pads or sponge pads, but are not so limited. When the operator desires to detach the base 722 from the casing 120, the operator simply has the stuck member 7224 of the top cover 7220 be released from the slot 7226 of the bottom cover 7222 and then rotates the top cover 7220 relatively to the bottom cover 7222, the casing is out of the base 722. Please not that the components shown in FIG. 9 with the same labeling numbers as those shown in FIG. 1 operate in substantially the same way and are not repeatedly described in details herein.

As described above, in this invention the passage used for transporting the ultrasound conductive medium is disposed on the casing of the ultrasound probe. Both the passage and the container which contains the ultrasound conductive medium are connected to the driving device. When the operator performs the ultrasound inspection by the ultrasound probe, the operator simply puts the passage of the ultrasound probe closed to the inspected portion of the body and operates the driving device to transport the ultrasound conductive medium from the container to the passage and then to squeeze the ultrasound conductive medium out of the passage. The ultrasound conductive medium squeezed out of the passage can then be applied on the inspected portion by the casing of the ultrasound probe directly for the ultrasound scanning over the inspected portion. When the ultrasound conductive medium applied on the inspected portion is not enough or when the operator wants to check other inspected portion, the operator only needs to operate the driving device again to transport the ultrasound conductive medium from the container to the passage and then to squeeze the ultrasound conductive medium out of the passage without putting down the ultrasound probe. For example, a pedal is connected to the driving device. When the operator steps on the pedal to control the driving device, the operator's hand which does not hold the ultrasound probe can operate or set up the ultrasound scanning device upon the operator's need. In this way, it not only is more convenient and simple to perform the ultrasound inspection, but also increases the inspection efficiency.

Those skilled in the art will readily observe that numerous modifications and alterations of the device and method may be made while retaining the teachings of the invention.

What is claimed is:

1. An ultrasound probe comprising:
A casing;
An ultrasound scanner, disposed in the casing; and
A passage, disposed on the casing and used for transporting an ultrasound conductive medium.

2. The ultrasound probe of claim 1, wherein the passage and the casing are formed integrally.

3. The ultrasound probe of claim 1, further comprising a connection mechanism, the connection mechanism comprising a base detachably connected to the casing, and the passage and the base being formed integrally.

4. The ultrasound probe of claim 3, wherein the casing has a slot, the connection mechanism further comprises a stuck member, a hinge and a torsion spring, the stuck member is hinged with the based via the hinge, the torsion spring is disposed on the hinge and contacts the base and the stuck member, respectively, when the base is connected to the casing, the stuck member is stuck with the slot, when the stuck member is pressed, the stuck member is released from the slot and the torsion spring is twisted, and when the stuck member is released, the torsion spring generates a torsion force to have the stuck member return to where the stuck member is originally disposed.

5. The ultrasound probe of claim 4, wherein the base comprises a stopper used to limit rotation of the stock member.

6. The ultrasound probe of claim 3, wherein the connection mechanism further comprises an elastic member disposed on the base, when the base is connected to the casing, the elastic member is clamped between the casing and the base.

7. The ultrasound probe of claim 3, wherein the base is detachably disposed on the casing by magnetism or screwing.

8. The ultrasound probe of claim 3, wherein the based is ring-shaped hollow, the connection mechanism further comprises a ring-shaped hollow air bag disposed in the base, after the casing passes through and is then surrounded by the ring-shaped hollow air bag and the ring-shaped hollow air bag is full of air, the casing is clamped in the base by the ring-shaped hollow air bag.

9. The ultrasound probe of claim 8, wherein the connection mechanism further comprises an elastic member disposed in an inner side of the ring-shaped hollow air bag, after the casing passes through and is then surrounded by the ring-shaped hollow air bag and the ring-shaped hollow air bag is full of air, the elastic member contacts the casing.

10. The ultrasound probe of claim 3, wherein the base is ring-shaped hollow, the connection mechanism further comprises a ring-shaped hollow elastic member disposed in the base, and the casing passes through and is then surrounded by the ring-shaped hollow elastic member so as to have the casing be clamped in the base by the ring-shaped hollow elastic member.

11. The ultrasound probe of claim 3, wherein the base comprises a top cover and a bottom cover, one end of the top cover is hinged with the bottom cover, the other end of the top cover is selectively stuck with or released from the bottom cover, the connection mechanism further comprises two elastic members disposed on an inner side of the top cover and an inner side of the bottom cover, respectively, when the casing is disposed between the top cover and the bottom cover and the top cover is stuck with the bottom cover, the casing is clamped between the top cover and the bottom cover by the two elastic members.

12. An ultrasound scanning system comprising:
An ultrasound scanning device;
An ultrasound probe, communicated with the ultrasound scanning device, the ultrasound probe comprising:
A casing;
An ultrasound scanner, disposed in the casing; and
A passage, disposed on the casing;
A container used for containing an ultrasound conductive medium; and
A driving device, connected between the passage and the container, used for transporting the ultrasound conductive medium from the container to the passage and squeezing the ultrasound conductive medium out of the passage.

13. The ultrasound scanning system of claim 12, wherein the passage and the casing are formed integrally.

14. The ultrasound scanning system of claim 12, wherein the ultrasound probe further comprises a connection mechanism, the connection mechanism comprises a base detachably connected to the casing, and the passage and the base are formed integrally.

15. The ultrasound scanning system of claim 14, wherein the casing has a slot, the connection mechanism further comprises a stuck member, a hinge and a torsion spring, the stuck member is hinged with the based via the hinge, the torsion spring is disposed on the hinge and contacts the base and the stuck member, respectively, when the base is connected to the casing, the stuck member is stuck with the slot, when the stuck member is pressed, the stuck member is released from the slot and the torsion spring is twisted, and when the stuck member is released, the torsion spring generates a torsion force to have the stuck member return to where the stuck member is originally disposed.

16. The ultrasound scanning system of claim 15, wherein the base comprises a stopper used to limit rotation of the stock member.

17. The ultrasound scanning system of claim 14, wherein the connection mechanism further comprises an elastic member disposed on the base, when the base is connected to the casing, the elastic member is clamped between the casing and the base.

18. The ultrasound scanning system of claim 14, wherein the base is detachably disposed on the casing by magnetism or screwing.

19. The ultrasound scanning system of claim 14, wherein the based is ring-shaped hollow, the connection mechanism further comprises a ring-shaped hollow air bag disposed in the base, the driving device is connected to the ring-shaped hollow air bag and used to activate air inflation or leakage of the ring-shaped hollow air bag, after the casing passes through and is then surrounded by the ring-shaped hollow air bag and the ring-shaped hollow air bag is full of air, the casing is clamped in the base by the ring-shaped hollow air bag.

20. The ultrasound scanning system of claim 19, wherein the driving device comprises a first pump and a second pump, the first pump is connected to the passage and the container for transporting the ultrasound conductive medium from the container to the passage and squeezing the ultrasound conductive medium out of the passage, and the second pump is connected to the ring-shaped hollow air bag to activate air inflation or leakage of the ring-shaped hollow air bag.

21. The ultrasound scanning system of claim 19, wherein the connection mechanism further comprises an elastic member disposed in an inner side of the ring-shaped hollow air bag, after the casing passes through and is then surrounded by the ring-shaped hollow air bag and the ring-shaped hollow air bag is full of air, the elastic member contacts the casing.

22. The ultrasound scanning system of claim 14, wherein the base is ring-shaped hollow, the connection mechanism further comprises a ring-shaped hollow elastic member disposed in the base, and the casing passes through and is then surrounded by the ring-shaped hollow elastic member so as to have the casing be clamped in the base by the ring-shaped hollow elastic member.

23. The ultrasound scanning system of claim 14, wherein the base comprises a top cover and a bottom cover, one end of the top cover is hinged with the bottom cover, the other end of the top cover is selectively stuck with or released from the bottom cover, the connection mechanism further comprises two elastic members disposed on an inner side of the top cover and an inner side of the bottom cover, respectively, when the casing is disposed between the top cover and the bottom cover and the top cover is stuck with the bottom cover, the casing is clamped between the top cover and the bottom cover by the two elastic members.
